(19) United States
(12) Patent Application Publication
(10) Pub. No.: US 2002/0132982 A1
(43) Pub. Date: Sep. 19, 2002

(54) AHF associated dispersion system and method for preparation

(76) Inventors: Sathyamangalam V. Balasubramanian, Amherst, NY (US); Marc Besman, Studio City, CA (US); Ramesh Kashi, Walnut, CA (US); Karthik Ramani, Amherst, NY (US)

Correspondence Address: Michael L. Goldman, NIXON PEABODY LLP, Clinton Square, P.O. Box 31051, Rochester, NY, 14603, US

(21) Appl. No.: 09/997,936

(22) Filed: Nov. 30, 2001

Related U.S. Application Data
(60) Provisional application No. 60/250,137, filed on Nov. 30, 2000.

(51) Int. Cl. C07K 1/00; C07K 14/00; C07K 17/00

(52) U.S. Cl. 530/350

(57) ABSTRACT

A method for complexing AHF in a dispersed medium, includes: a) providing an AHF protein, b) altering the conformational state of the AHF protein to expose hydrophobic domains therein, c) binding a stabilizer to the exposed hydrophobic domains, and d) at least partially reversing the alteration to associate at least a portion of the protein with the stabilizer. A pharmaceutically effective stabilized AHF dosage wherein above about 0.5%, preferably above about 3%, and more preferably above about 25%, of the AHF molecule is associated with a stabilizer is also disclosed.

### BACKGROUND OF THE INVENTION

This application claims the benefit of U.S. Provisional Patent Application Serial No. 60/250,137, filed Nov. 30, 2000 (which is hereby incorporated by reference in its entirety).

### FIELD OF THE INVENTION

The present invention relates to Antihemophilic (“AHF”) protein associated with a dispersion system and methods for preparation thereof. In particular, the present invention relates to liposomal association, including encapsulation, of AHF proteins and methods for preparation thereof.

### DESCRIPTION OF THE RELATED ART

Hemophilia A is an inherited bleeding disorder caused by deficiency of antihemophilic factor, a blood coagulation protein that functions as a cofactor in the coagulation cascade (Lamer, A. J. (1987) “The molecular pathology of haemophilia.” Q J Med 63(242): 473-91, which is hereby incorporated by reference in its entirety). It is transmitted as a defect on the X chromosome and affects 1 male in 5,000. The severity of the bleeding disorder varies among patients depending upon the degree of deficiency of AHF.

Recent advances in biotechnology and protein engineering, together with cloning of the gene coding for AHF (Toole, J. J., J. L. Knopf, et al. (1984) “Molecular cloning of a cDNA encoding human antihaemophilic factor.” Nature 312(5992): 342-7; Wood, W. I., D. J. Capon, et al. (1984) “Expression of active human factor VIII from recombinant DNA clones.” Nature 312(5992): 330-7, which are hereby incorporated by reference in their entirety), have made it feasible to manufacture recombinant human AHF (“rhAHF”). The recombinant preparation promises to be a source of unlimited supply, together with the freedom from the complications of transmission of blood-borne viruses. However, the commercially available protein pharmaceutical has been reported to undergo the aforementioned physical instability problems with concomitant loss of therapeutic activity (Baxter Health Care, unpublished results), thus requiring a new formulation strategy. This requires an understanding of its complex structure and dynamic folding features. Structurally, the AHF can be divided into a heavy chain, including domains A1 and A2, and a light chain, including domains A3, C1 and C2. The heavy and the light chains are connected in space by a third distinct B domain (Fay, P. J. (1993) “Factor VIII structure and function.” Thromb Haemost 70(1):63-7; Foster, P. A. and T. S. Zimmerman (1989) “Factor VIII structure and function.” Blood Rev 3(3): 180-91, which are hereby incorporated by reference in their entirety) However, the important features related to the folding behavior of this large multi-domain protein are unknown in the art, and may be an integral part of an effective approach towards development of a stable formulation.

Recombinant human factor VIII is a multi-domain protein used in the treatment of patients with classical Hemophilia A. Because of its complex structure and folding characteristics, the present clinical formulation has several disadvantages; (1) formulations show physical instability, denaturation of the protein promoting aggregation that is associated with the concomitant loss of activity (2) formulations have a short half life in circulation, requiring frequent administration of the drug and (3) formulations illicit immune response requiring higher doses of frequent administration. This translates into not only higher cost but also patient discomfort.

Thus, there is a need for suitable AHF-based pharmaceuticals having improved stability during processing and storage conditions; increased dosage spacing by increasing bioavailability, thus reducing cost and patient discomfort; easy handling; and improved delivery to site of vascular damage.

The present invention is directed to overcoming these and other deficiencies in the art.

### SUMMARY OF THE INVENTION

The present invention relates to a method for associating AHF in a dispersed medium, including: a) providing an AHF protein, b) altering the conformational state of the AHF protein to expose hydrophobic domains therein, c) binding a stabilizer to the exposed hydrophobic domains, and d) at least partially reversing the alteration to associate at least a portion of the protein with the stabilizer.

Another object of the present invention is to provide a pharmaceutically effective stabilized AHF dosage wherein above about 0.5%, preferably above about 3%, and more preferably above about 25% of the AHF is associated with a stabilizer.

These and other aspects of the present invention will become apparent upon a review of the following detailed description and the claims appended thereto.

### BRIEF DESCRIPTION OF THE DRAWINGS

FIG. 1A is a graph of the far UV-CD spectra of the molar ellipticity of rhAHF in the appropriate buffer acquired between 20° C. and 90° C. over a wavelength range of from 208 nm to 255 nm. FIG. 1B is a graph of the near-UV CD spectra of the molar ellipticity of rhAHF in the appropriate buffer acquired between 20° C. and 80° C. over a wavelength range of from 255 nm to 320 nm. FIG. 1C is a graph of the ellipticity of rhAHF at 215 nm and 295 nm as a function of temperature.

FIG. 2 is a graph of the temperature dependent changes in fluorescence intensity, measured in arbitrary units (“a.u.”), of rhAHF.

FIG. 3 is a graph of the % change in ANS fluorescence as a function of temperature.

FIG. 4 is a graph of the ellipticity of rhAHF as a function of temperature.

FIG. 5 is a graph of the molar ellipticity of rhAHF and rhAHF in liposomes.

FIG. 6A is a graph showing the controlled heating and recooling of rhAHF in the appropriate buffer carried out over the temperature range of from 20° C. to 90° C. in the absence of liposomes. FIG. 6B is a graph showing the controlled heating and recooling of rhAHF in the appropriate buffer carried out over the temperature range of from 20° C. to 90° C. in the presence of liposomes.

FIG. 7 is a schematic representation of the folding characteristics of rhAHF and its relation to physical instability pathways.

FIG. 8 is a schematic representation of the rhAHF-liposome complex based delivery vehicle of the present invention.

FIG. 9 is a graph showing the effect of liposomes on the secondary structure of rhAHF.

### DETAILED DESCRIPTION OF PREFERRED EMBODIMENTS

In order to overcome the above-noted problems, the present invention provides a method wherein the Antihemophilic protein is associated with the dispersion system, such as liposomes, through complex association, including encapsulation by the liposomes.

This present methodology of liposomal association of AHF proteins for use in the delivery of protein derived products offers several advantages: improvement in stability; prolonging of the circulation time; and alteration of the immunogenicity issues. In particular, this invention can be effectively used with AHF formulations to provide dispersion systems as a delivery vehicle for AHF and its fragments. The present invention provides: (1) a novel complex AHF-based delivery vehicle; (2) since AHF is a multidomain protein several intermediates are possible and the association of a specific intermediate may be advantageous over another, most likely because the association of a specific region of the protein may influence several aspects, such as: alter protein refolding, and provide less immune response due to the association of the C2 domain; (3) the use of a combination of perturbants, solvent and heat, to enhance the effect and achieve exposure at lower temperatures; and (4) stabilization of the AHF complex with hydrophobic domain interaction.

Since the process of denaturation is related to an alteration in the conformational state of the protein antihemophilic factor (AHF/factor VIII), used in the treatment of Hemophilia A, such as unfolding at the molecular level, we analyzed the conformational changes as the protein unfolds and applied novel methods at key steps in the process. In accordance with the present invention, we have carried out biophysical studies to understand the conformational changes, e.g., folding/unfolding properties of rhAHF in detail, with specific experimental approaches to investigate the existence of intermediate structures and their role in aggregation.

Our interest in using liposomes as an AHF formulation excipient is three-fold. (1) Liposomes have previously been shown to stabilize protein against aggregation and act as molecular chaperones, altering the protein-folding pathway (Yoshimoto, M. and R. Kuboi (1999) “Oxidative refolding of Denatured/Reduced lysozyme utilizing the chaperone-like function of liposomes and immobilized liposome chromatography.” Biotechnol Prog 15(3): 480-7; Balasubramanian, S. V., J. Bruenn, et al. (2000) “Liposomes as formulation excipients for protein pharmaceuticals: a model protein study [In Process Citation].” Pharm Res 17(3): 344-50.), which are hereby incorporated by reference in their entirety). (2) Surface modified liposomes have been shown to alter the circulation time of protein therapeutics, making them attractive delivery vehicles for biotechnology-derived products (Kanaoka, E., S. Nagata, et al. (1999) “Stabilization of aerosolized IFN-gamma by liposomes.” Int J Pharm 188(2): 165-72.); Woodle, M. C. (1993) “Surface-modified liposomes: assessment and characterization for increased stability and prolonged blood circulation.” Chem Phys Lipids 64(1-3): 249-62, which are hereby incorporated by reference in their entirety). It has been documented that the AHF binds strongly to PS containing phospholipids (Gilbert, G. E., D. Drinkwater, et al. (1992) “Specificity of phosphatidylserine-containing membrane binding sites for factor VIII. Studies with model membranes supported by glass microspheres (lipospheres).” J Biol Chem 267(22): 15861-8; Gilbert, G. E. and D. Drinkwater (1993) “Specific membrane binding of factor VIII is mediated by O-phospho-L-serine, a moiety of phosphatidylserine.” Biochemistry 32(37); 9577-85, which are hereby incorporated by reference in their entirety) in vivo. In the present invention, we have examined the interaction of the protein with PS containing liposomes to develop alterations in the protein-folding pathway and exploited such alterations in the development of the delivery vehicle of the present invention. (3) We have used liposomes to shield the epitope regions, such as C2, to reduce immune response and antigenicity by lipid binding.

In accordance with the present invention, we have used liposomes as an excipient to stabilize the AHF protein against the physical instability. Circular Dichroism (“CD”) and fluorescence spectroscopy was used to study the temperature dependent folding/unfolding characteristics of the protein in the presence and absence of liposomes. ANS (“1,8 anilininaphthalene sulfonate”), a fluorescent probe that partitions into hydrophobic domains, was used to detect exposure of the AHF hydrophobic domains. CD studies indicated that, at elevated temperature (about 60° C.), the protein appeared to retain most of its secondary structural features but lost its tertiary structure suggesting the existence of partially folded, structured intermediates. The unfolding of the protein was associated with the exposure of hydrophobic domains as observed by ANS fluorescence. These intermediate states with exposed hydrophobic domains may be involved in the reported aggregation associated with loss of activity and the aggregates are possibly stabilized by intermolecular β-strands. We have found through equilibrium refolding studies that the thermally denatured protein in the presence of liposomes follows a different folding pathway and may be due to the interaction of the liposomes with the AHF protein, possibly resulting in the formulation of a complex. The ramification of the present observation is that, this protein-liposome complex can be exploited as a delivery vehicle, not only to improve stability of factor VIII but also to prolong its circulation time in vivo.

In this invention, we have developed a liposome-containing AHF formulation to provide stability, improve the circulation time of the protein and reduce immune responses, thus overcoming the problems associated with present clinical formulations. For the purposes of the present invention, liposomes are defined as microcapsules having a hydrophobic interior and a hydrophillic exterior synthesized from lipids. Other suitable dispersed systems include micelles, detergents, and the like.

Thus, an important aspect of this invention is the lipid-AHF protein complex based delivery vehicle. Optionally, we can coat this lipid-protein association with molecules such as Poly ethylene Glycol (PEG) to provide stealth properties to the delivery vehicle.

In this invention, we achieved the liposome based formulation by engineering a complex formation between AHF and the phosphatidyl serine (PS) head group containing liposomes. We also stabilized this complex through hydrophobic interactions between liposomes and specific intermediate states in the protein, exploiting the chaperone like molecular characteristics of the lipid assembly. The complexed intermediate also aids in refolding of the protein to its native state. The association of the liposome with the AHF protein resulted in increased stability, preventing aggregation. The present invention enables the encapsulation of above about 3% of the protein, preferably above about 25% (+/− about 3%) of the AHF protein. The present invention enables the association of above about 0.5%, preferably above about 3%, more preferably above about 25%, and most preferably above about 75% of the AHF protein with a stabilizer.

TABLE 1
Percent AHF protein associated using
conventional and present technology.

| Sample | % Protein associated |
|---|---|
| Conventional method | 49 |
| Present methodology | 71.3 |

The liposome loaded protein was separated from the free factor VIII by rapid sedimentation as describe previously (Gary Gilbert, Barbara Furie and Bruce Furie, Binding of Human factor VIII to phospholipid vesicles, J.Biol.Chem 266, 815-822 (1990), which is hereby incorporated by reference in its entirety). The complex was spun at a rate of 15,000 g at 4° C. for 45 minutes. The supernatant and the resuspended pellets were analyzed by fluorescence spectroscopy. The unbound protein was separated from liposome bound protein by dextran centrifugation gradient. The samples were spun at 185,000 g for 30 min using SW50.1 Beckman rotor. The samples were analyzed by fluorescence and activity assay to determine the encapsulation efficiency.

The molecular association prepared in accordance with the present invention showed comparable activity to present conventionally administered formulations as shown by the activity data in Table 2.

TABLE 2
Activity data

| | Concentration based on |
|---|---|
| Sample | activity (units/ml) |
| Free AHF Protein | 23.1 |
| Protein + liposome (conventional method) | 16.3 |
| Protein + liposome (present methodology) | 21.6 |

In order to increase the bioavailability of AHF, we coated this complex/delivery vehicle with polyethylene glycol, using PEG-phosphatidyl ethanolamine as one of the lipid components in liposomes. This also addresses the immunogenicity problems by providing stealth like properties to the protein-lipid complex.

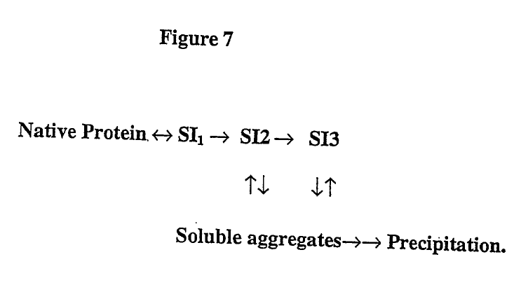

It has been documented that partial unfolding of the protein results in increased exposure of hydrophobic domains and promotes aggregation (Balasubramanian, S. V., J. Bruenn, et al. (2000) “Liposomes as formulation excipients for protein pharmaceuticals: a model protein study [In Process Citation].” Pharm Res 17(3): 344-50, which is hereby incorporated by reference in its entirety). Hence, thermal denaturation studies of rhAHF were carried out to understand the subtleties involved in the unfolding process and its contribution towards physical destabilization. The melting of the protein was a multistage process and small changes observed in the secondary structure coupled with progressive loss of tertiary structure as the unfolding proceeded, suggested the transition of the native conformation of the protein to structured intermediate state(s). The data here suggests the existence of at least three “structured intermediate” states and such changes can cause protein inactivation by promoting aggregation. Further, analysis based on the equilibrium refolding studies indicated that while the formation of the intermediate state SI₁ was reversible, appearance of SI₂ and SI₃ in the unfolding pathway resulted in the protein loosing its structure irreversibly (data not shown). The above observations suggest that, intermediate state(s) formation occurring after or coinciding with the first major transition could possibly result in irreversible loss of protein structure (as shown in the schematic representation if FIG. 7) leading to precipitation of the protein.

The conformation of the AHF protein as determined by fluorescence spectroscopy indicates that sufficient exposure of hydrophobic domains occur in the temperature range of from 50° C. to 65° C. Such exposure can accelerate the aggregation process. Also, CD data indicate that the formation of intermolecular β-strands occurs at elevated temperatures. These observations lead us to hypothesize that the aggregates could possibly be stabilized by intermolecular β-strands and such stabilization in turn could promote precipitation over a period of time. Indeed, it has been documented that intermolecular β-strands mediate physical instability in proteins (Hilbich, C., B. Kisters-Woike, et al. (1991) “Aggregation and secondary structure of synthetic amyloid beta A4 peptides of Alzheimer's disease.” J Mol Biol 218(1):149-63; Hammarstrom, P., M. Persson, et al. (1999) “Structural mapping of an aggregation nucleation site in a molten globule intermediate.” J Biol Chem 274(46):32897-903, which are hereby incorporated by reference in their entirety).

A very common approach to counter physical instability problem in proteins has been to use excipients (Tsai, P. K., D. B. Volkin, et al. (1993) “Formulation design of acidic fibroblast growth factor.” Pharm Res 10(5): 649-59; Carpenter, J. F., M. J. Pikal, et al. (1997) “Rational design of stable lyophilized protein formulations: some practical advice.” Pharm Res 14(8): 969-75, which are hereby incorporated by reference in their entirety). However, the typical excipients used have been chosen empirically and the rationale underlying the formulation design assumes protein unfolding to be a two stage process, i.e. at any chosen time in the unfolding process, there exist only two kind of population; folded and unfolded molecules. While this might be valid for small proteins such as lysozyme and cytochrome C, such an assumption is an over simplification of the complex nature of the problem for multi-domain proteins like rhAHF. The studies of the present invention clearly suggest a multistage unfolding process and this can have profound implications. We have found that the choice of an excipient should be based on its preferential interaction to the partially folded structures as such interactions can possibly enhance the ability of the excipient to guide these structures back to the native state.

Liposomes have previously been shown to stabilize protein against aggregation by preferentially interacting with the intermediate structures (Balasubramanian, S. V., J. Bruenn, et al. (2000) “Liposomes as formulation excipients for protein pharmaceuticals: a model protein study [In Process Citation].” Pharm Res 17(3): 344-50, which is hereby incorporated by reference in its entirety). As AHF has been shown to bind strongly to PS containing phospholipids in vivo, PS containing liposomes were used to characterize the interaction with the protein. It has been documented that the presence of PS in membranes is essential for mediation of AHF binding (Gilbert, G. E. and Drinkwater, D. (1993) “Specific membrane binding of factor VIII is mediated by O-phospho-L-serine, a moiety of phosphatidylserine.” Biochemistry 32(37): 9577-85, which is hereby incorporated by reference in its entirety). Also, the PS binding site has been localized to the C2 domain (which is part of the light chain) of AHF, though not much is known about the spatial orientation of the other five domains (Foster, P. A., C. A. Fulcher, et al. (1990) “Synthetic factor VIII peptides with amino acid sequences contained within the C2 domain of factor VIII inhibit factor VIII binding to phosphatidylserine.” Blood 75(10): 1999-2004, which is hereby incorporated by reference in its entirety). However, based on a model proposed for the interaction of coagulation factor V (fV) (Gilbert, G. E., B. C. Furie, et al. (1990) “Binding of human factor VIII to phospholipid vesicles.” J Biol Chem 265(2): 815-22; Kalafatis, M., M. D. Rand, et al. (1994) “Factor Va-membrane interaction is mediated by two regions located on the light chain of the cofactor.” Biochemistry 33(2): 486-93; Lecompte, M. F., G. Bouix, et al. (1994) “Electrostatic and hydrophobic interactions are involved in factor Va binding to membranes containing acidic phospholipids.” J Biol Chem 269(3): 1905-10, which are hereby incorporated by reference in their entirety), (a rhAHF homologue) with phospholipid, it has been proposed that regions other than C2 domain sequence may also be involved in membrane binding (Saenko, E. L., Scandella, D. (1995) “A mechanism for inhibition of factor VIII binding to phospholipid by von Willebrand factor,” J Biol Chem 270(3):13826-13833, which is hereby incorporated by reference in its entirety). Though the exact nature of this interaction is not known we envision that PS containing liposomes interact with the protein, inducing conformational changes, resulting in the intercalation of the hydrophobic regions of C2 domain of the protein into the bilayer membrane. The polar head groups probably interact with the hydrophilic regions offering additional stability to the complex. Further, we speculate that the liposomes may provide a hydrophobic environment to the unfolding protein, thereby interacting preferentially with the partially folded structures and preventing intermolecular association. Initial spectral characterization studies suggest that the interaction of the liposomes with the protein appears to be dependent on the nature of the intermediate state as reflected in the reversibility associated with these intermediate states. In the presence of liposomes, while the formation of SI₁ was completely reversible, appearance of SI₂ resulted in irreversible loss of protein structure. However, there was partial recovery of the native-like features following formation of SI₃ (data not shown). We also reason that the association with the PS containing liposomes induces the native structure of the protein to adopt a “non-native” like conformation and probably plays an important role in the unfolding/refolding process. Assuming that the protein sub-units in the heavy and light chains melt in a particular sequence during the denaturation process, in the event of liposomes binding to C2, this sequential melting of the sub-units might be altered. This is reflected in the observation from our fluorescence (data not shown) and far-UV CD studies that suggests that the protein, in the presence of liposomes appear to take a different unfolding/refolding pathway. If this is true, then the lipid molecules associated with the protein might also be involved in guiding the protein towards its native state. Nevertheless, irrespective of the nature of the complex, this study gives a perspective to address the formulation issues concerning this complex protein and is discussed in the following section.

It has been reported that the C2 domain of the protein is involved in high affinity interaction with the phospholipids present on the surface of the platelets (Foster, P. A., C. A. Fulcher, et al. (1990) “Synthetic factor VIII peptides with amino acid sequences contained within the C2 domain of factor VIII inhibit factor VIII binding to phosphatidylserine.” Blood 75(10):1999-2004, which is hereby incorporated by reference in its entirety). Further, it has been shown inhibitors against the C2 domain are primarily responsible for neutralizing AHF activity in vivo (Scandella, D. H., Nakai, H. et al. (2001) “In Hemophilia A and Autoantibody Inhibitor Patients: The Factor VIII A2 Domain and Light Chain are Most Immunogenic,” Throm. Res., 377-385, which is hereby incorporated by reference in its entirety). The lipidic rAHF dispersions produced in this invention shield the epitope regions of the protein to reduce immune response and the antigenicity. In order to determine specifically whether the C2 domain is shielded in liposomes, an ELISA (“enzyme-linked immunosorbent assay”) using C2 domain specific antibodies was prepared. Protein bound liposomes were coated onto Nunc Maxicorp plates by incubating overnight at 4° C. in carbonate buffer. The antibody binding (as measured by OD) was found to be far less for the liposome bound protein than for free protein, indicating the epitope region is not available for the antibody binding. In addition to ELISA, fluorescence quenching by acrylamide was carried out to determine the accessibility of fluorophore on the protein to collisional quenchers. This data would provide information on the location of the protein in liposomes. The quenching experiment indicated that the lipidic rhAHF produced by this procedure indicated that the quenching efficiency of 0.2M acrylamide is 38% lower for liposome bound protein than that for the free protein. The results support the ELISA assay in which the part of the protein molecule is shielded as a result of lipid binding.

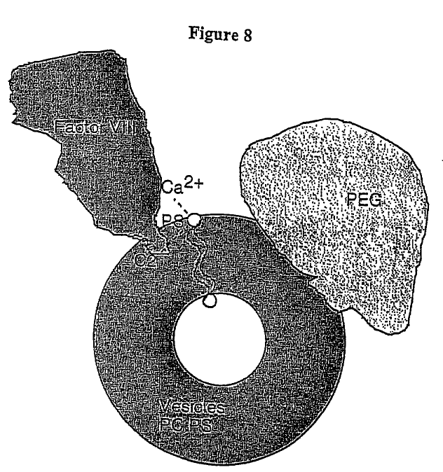

The above observations illustrate the desirable features of using liposomes as a stabilizer in rhAHF formulations. The complex formation between the protein and lipid may further enhance the stability of the protein in vivo by preventing enzyme(s) inactivation. Further, surface modified liposomes can also be used as a safe delivery vehicle (Woodle, M. C., K. K. Matthay, et al. (1992) “Versatility in lipid compositions showing prolonged circulation with sterically stabilized liposomes.” Biochim Biophys Acta 1105(2): 193-200, which is hereby incorporated by reference in its entirety). One of the approaches to achieve this has been to coat the surface with hydrophilic polymers such as polyethyleneglycol (PEG) (Klibanov, A. L., K. Maruyama, et al. (1990) “Amphipathic polyethyleneglycols effectively prolong the circulation time of liposomes.” FEBS Lett 268(1): 235-7, which is hereby incorporated by reference in its entirety). PEGylated liposomes have also been shown to circulate in the blood for a longer time, by evading the RES system (Lasic, D. D., Martin, F. J. et al. (1991) “Sterically stabilized liposomes: a hypothesis on the molecular origin of the extended circulation times.” Biochim Biophys Acta 1070(1): 187-92; Papahadjopoulos, D., Allen, T. M. et al. (1991) “Sterically stabilized liposomes: improvements in pharmacokinetics and antitumor therapeutic efficacy.” Proc Natl Acad Sci USA 8(24): 11460-4, which are hereby incorporated by reference in their entirety). We have explored the feasibility of using PEGylated liposomes as a means to (1) promote complex formation and stabilize the protein (FIG. 8) and (2) as a vehicle to deliver the rhAHF in vivo thereby increasing its circulation time. Preliminary studies have shown that the activity of the protein associated with liposomes is comparable to that of the free protein (data not shown). Heating in the presence of the solvent will expedite the desired conformational changes required for the liposomal association of the AHF protein.

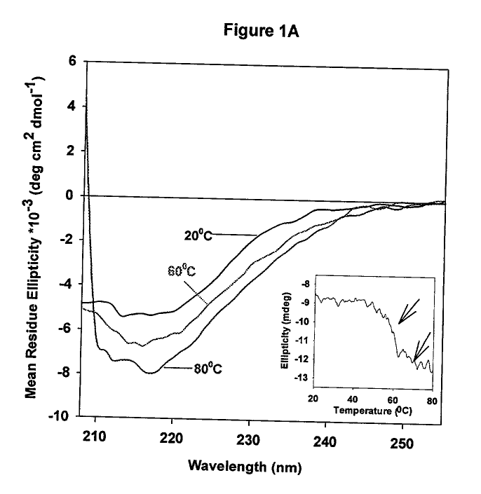

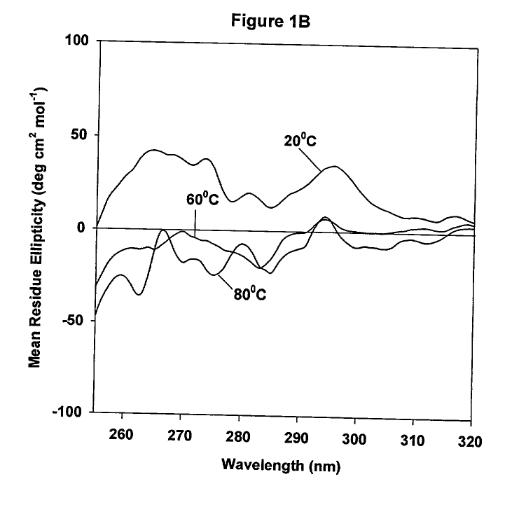

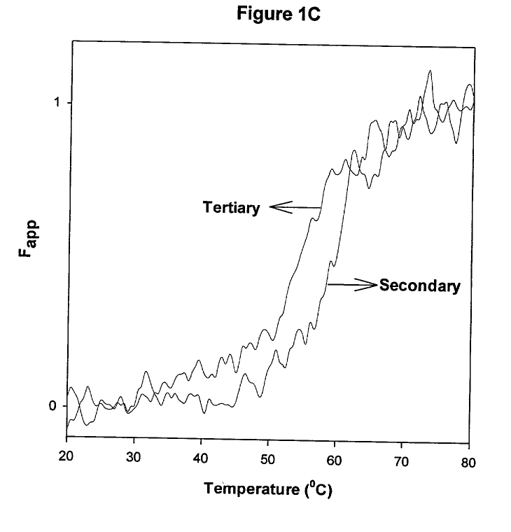

FIGS. 1A-C relate to the temperature dependence of the secondary and tertiary structures of rhAHF. FIG. 1A is a graph of the far UV-CD spectra of the molar ellipticity of rhAHF in the appropriate buffer acquired between 20° C. and 90° C. over a wavelength range of from 208 nm to 255 nm. The path length of the quartz cuvette used was 10 mm and the concentration of the protein was 20 μg/ml. FIG. 1B is a graph of the near-UV CD spectra of the molar ellipticity of rhAHF in the appropriate buffer acquired between 20° C. and 80° C. over a wavelength range of from 255 nm to 320 nm. The path length of the quartz cuvette used was 10 mm and the concentration of the protein was 0.5 mg/ml. FIG. 1C is a graph of the temperature dependence of the secondary and tertiary structures of rhAHF. The temperature dependent changes in the secondary and tertiary structures of water rhAHF in the appropriate buffer are compared by plotting the ellipticity at 215 nm and 295 nm as a function of temperature. Fₐₚₚ, the fraction of protein in the unfolded state, was calculated as described in the experimental procedures noted below.

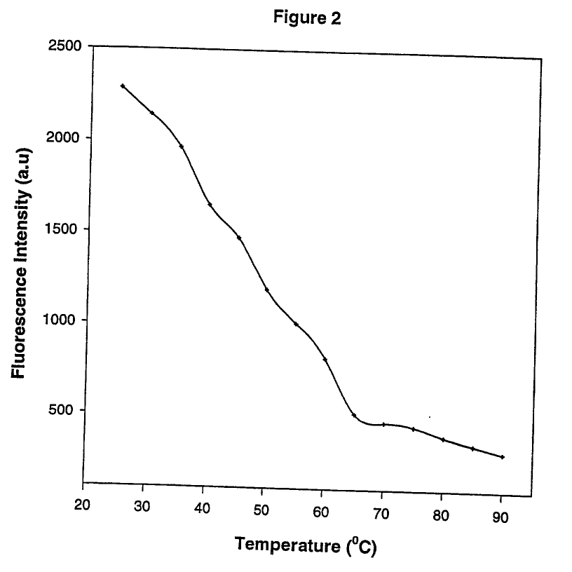

FIG. 2 is a graph of the temperature dependent changes in fluorescence intensity, measured in arbitrary units (“a.u.”), of rhAHF. Fluorescence emission spectra of rhAHF in the appropriate buffer were acquired over the temperature range of from 20° C. to 90° C., in 5° C. increments. The excitation wavelength was 280 nm and the emission was monitored at 330 nm. The emission and excitation slits used were 4 nm. The path length of the cuvette used was 10 mm and the concentration of the protein was 10 nM.

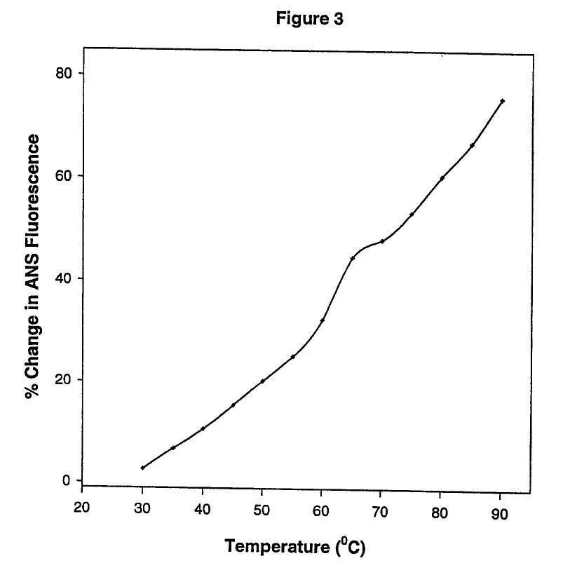

FIG. 3 is a graph of the % change in ANS fluorescence as a function of temperature. The exposure of hydrophobic domains of rhAHF was probed by ANS complex formation. ANS was dissolved at high concentrations in water and a small volume was added to 10 nM rhAHF, to a final probe concentration of 50 nM. The sample was excited at 380 nm and the emission was monitored at 482 nm. Each data point is an average of three experiments.

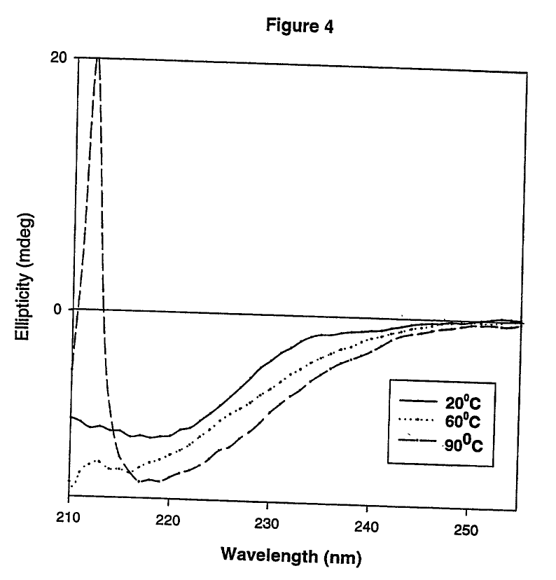

FIG. 4 is a graph of the ellipticity of rhAHF as a function of temperature. The formation of intermolecular β-strands in rhAHF following incubation was measured at various temperatures. The rhAHF was incubated for about 5 to 7 minutes at the indicated temperatures and far-UV CD spectra were acquired over the range of from 210 nm to 255 nm using a 10 mm path length quartz cuvette.

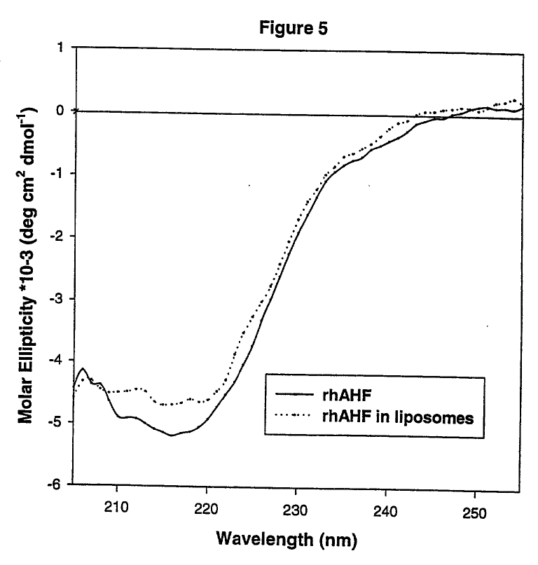

FIG. 5 is a graph of the molar ellipticity of rhAHF and rhAHF in liposomes as a function of temperature. FIG. 5 shows the effect of liposomes on the secondary structure of rhAHF: Far-UV CD spectra of rhAHF were acquired in the presence and absence of multilamellar liposomes composed of DMPC and bPS. The protein concentration was 20 μg/ml and the protein/lipid ratio was about 1:6000. CD spectrum was acquired over a range of from 205 nm to 255 nm using a 10 mm path length quartz cuvette. Sample preparation is described below.

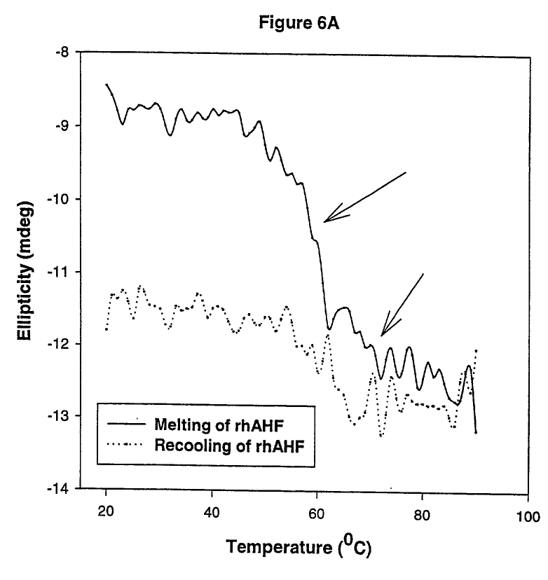

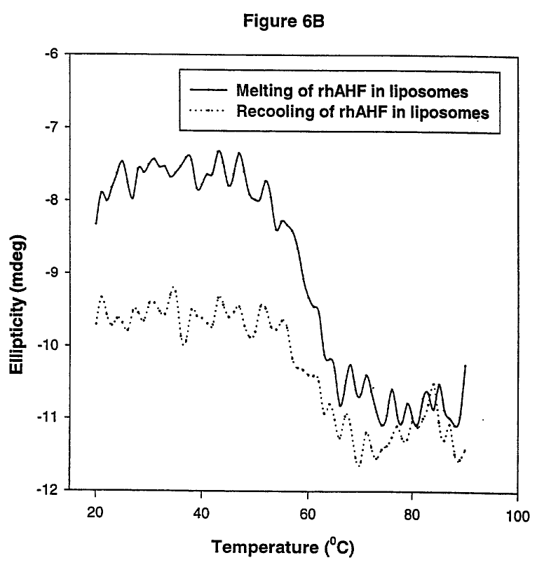

FIGS. 6A-B show equilibrium refolding of rhAHF in the presence and absence of liposomes. FIG. 6A is a graph showing the controlled heating and recooling of rhAHF in the appropriate buffer carried out over the temperature range of from 20° C. to 90° C. in the absence of liposomes. Typically the concentration of the protein used was about from 20 μg/ml to 22 μg/ml. FIG. 6B is a graph showing the controlled heating and recooling of rhAHF in the appropriate buffer carried out over the temperature range of from 20° C. to 90° C. in the presence of liposomes. Typically, the protein concentration was from about 20 μg/ml to 22 μg/ml and the protein/lipid ratio was about 1:6000. Sample preparation is described below.

FIG. 7 is a schematic representation of the folding characteristics of rhAHF and its relation to physical instability pathways.

FIG. 8 is a schematic representation of the rhAHF-liposome complex based delivery vehicle of the present invention.

### EXAMPLES

The invention will be illustrated in greater detail by the following specific examples. It is understood that these examples are given by way of illustration and are not meant to limit the disclosure or the claims to follow.

### Materials

The rhAHF was provided by Baxter Health Care, CA and was used without further purification. ANS (1-anilino-8 naphthalene sulfonate), a hydrophobic probe (Aloj, S. M., K. C. Ingham, et al. (1973) “Interactions of 1,8 ANS with human luteinizing hormones: A probe for subunit interactions of hcg and hlh.” Arch. Biochem. Biophysics 155: 478-479; Purohit, S., K. Shao, et al. (1997) “Mutants of human choriogonadotropin lacking N-glycosyl chains in the alpha-subunit. 1. Mechanism for the differential action of the N-linked carbohydrates.” Biochemistry 36(40): 12355-63; Balasubramanian, V., L. T. Nguyen, et al. (1998) “Interferon-gamma-inhibitory oligodeoxynucleotides alter the conformation of interferon-gamma.” Mol Pharmacol 53(5):926-32, which are hereby incorporated by reference in their entirety) was purchased from Molecular Probes Inc (Eugene Oreg.). Lipids were obtained from Avanti polar lipids, AL and used without purification.

### Example 1

### Preparation of Liposomes

### Conventional Method

In the procedure according to the conventional method: 0.30 mg/ml DMPC, 0.15 mg/ml bPS and 0.04 mg/ml DSPE-PEG dissolved in chloroform were taken in a round-bottomed flask and the solvent was removed using a rotary evaporator, depositing the lipid as a thin film along the walls of the flask. Multilamellar vesicles (MLV) encapsulating the protein were formed by dispersing the lipid film in the appropriate buffer (0.4 M NaCl and 50 Mm Tris) containing 0.5 mg/ml of the protein, with gentle swirling at room temperature.

### Present Methodology

### Method 1

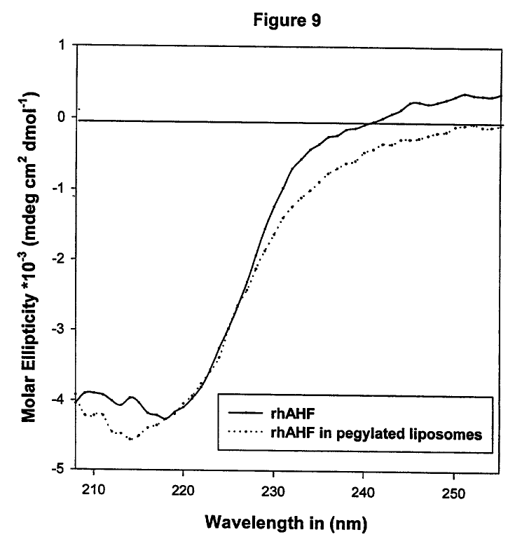

In the procedure according to the present method: 0.30 mg/ml DMPC, 0.15 mg/ml bPS and 0.04 mg/ml DSPE-PEG dissolved in chloroform were taken in a round-bottomed flask and the solvent was removed using a rotary evaporator, depositing the lipid as a thin film along the walls of the flask. Multilamellar vesicles (MLV) encapsulating the protein were formed by dispersing the lipid film in the appropriate buffer (0.4 M NaCl and 50 Mm Tris and ˜10%v/v ethanol) containing 0.5 mg/ml of the protein, with gentle swirling at room temperature or at 37° C. The protein stock solution was prepared by adding 50 ml of 95% ethanol to 450 ml of the protein solution in the specified buffer. Ethanol was then removed using a liquid nitrogen flow at room temperature or at about 37° C. A graph of data generated from this example showing the effect of liposomes on the secondary structure of rhAHF: Far-UV CD spectra of rhAHF were acquired in the presence and absence of PEGylated liposomes composed of DMPC (dimyrsitolphosphatidyl choline), bPS (brain phosphatidylserine) and DSPE-PEG (distearoyl phosphatidyl ethanolamine-Polyethylene Glycol-2000) is shown in FIG. 9.

### Method 2

In the present invention several preparation procedures were attempted to achieve the goals. 0.3 mg/ml DMPC, 0.15 mg/ml bPS, cholesterol dissolved in chloroform were taken in a round bottomed flask and the solvent was removed using a rotary evaporator, depositing the lipid as a thin film along the walls of the flask. The MLVs thus formed were filtered through a polycarbonate filter (0.22 μm) to form SUVs below 200 nm. The liposomes encapsulating the protein were formed by mixing the liposomes in protein containing buffer and ethanol followed by gentle swirling at 37° C. or at higher temperatures to generate intermediate structures. The average diameter of the particles was 160 nm. The PEGyation of these particles were performed by adding DSPE-PEG.

### Example 2

### Circular Dichroism Experiments

CD spectra were acquired on a JASCO-715 spectropolarimeter calibrated with d10 camphor sulfonic acid. Samples were scanned in the range of from 205 nm to 255 nm for secondary structure analysis, and typically, the protein concentration used was about from 20 μg/ml to 22 μg/ml. For near-UV CD studies, spectra were acquired in the range of from 320 nm to 255 nm, using a 10 mm quartz cuvette, and the protein concentration used was about 0.5 mg/ml. CD spectra of the protein were corrected by subtracting the spectrum of the buffer baseline and multiple scans were acquired and averaged to improve signal quality. The CD spectra of samples containing liposomes may be distorted as a result of light scattering. The contribution due to light scattering was corrected as follows: (1) the ellipticity values at from 350 nm to 400 nm were monitored and used as a baseline that was subtracted from the scans; (2) multiple scans were acquired and averaged to improve the signal quality. The spectra thus obtained were invariant with the path length of the cuvette, dilution of the sample or position of the sample along the light path, indicating that the effect of scattering on the spectra was minimal.

### Example 3

### Fluorescence Studies

Fluorescence spectra were acquired on a SLM 8000C spectrofluorometer (Urbana, Ill.). The intensity of the emission spectra was monitored over the range of from 300 nm to 400 nm, using a slit width of 4 nm on the excitation and emission paths. The excitation monochromator was set at 280 nm and a 295 nm long pass filter was used to minimize scattering effects. The melting of the protein was followed by monitoring the decrease in the intensity of the emission at 330 nm over the temperature range of from 25° C. to 90° C. Samples were equilibrated at the desired temperature for approximately 3 to 4 minutes using a water bath (Neslab RTE 110).

### Example 4

### Equilibrium Folding Analysis

A two-state unfolding model was used to analyze the equilibrium unfolding data. To compare the transitions detected by several methods, each unfolding curve was normalized to the apparent fraction of the unfolded form (Fₐₚₚ), using the relationship:

Fₐₚₚ=(Y_{obs}-Yₙₐₜ)/(Y_{unf}-Yₙₐₜ)

Where Y_{obs} is the molar ellipiticity (at 215 nm or 295 nm) at a given temperature, and Y_{unf} and Yₙₐₜ are the spectral values for unfolded and native structures, respectively. Y_{unf} and Yₙₐₜ are obtained by taking the average of the spectrum plateau region at high and low temperatures, respectively.

### Example 5

### ANS Binding Studies

ANS (1-anilino-8-napthalene sulfonate) was dissolved in high concentration in the appropriate buffer and a small volume was added to a solution of 10 nM/ml rhAHF, to give a final probe concentration of 50 nM/ml. The excitation wavelength was 380 nm and the emission was monitored at 482 nm. Correction for the inner filter effect was performed by appropriate procedures as described above (Lakowicz, J. R., Ed. (1986) Principles of Fluorescence Spectroscopy, New York, Plenum Press, which is hereby incorporated by reference in its entirety).

### Example 6

### Biological Activity Assay of rhAHF

RhAHF clotting activity was determined by a one-stage activated partial thromboplastin time (APTT) assay using micronized silica or CaCl₂ as an activator and AHF deficient plasma as the substrate (Kashi et al unpublished results). The APTT assay was performed using a COAG-A-MATE coagulation analyzer (Organon Teknika Corporation, Durham, N.C.). Briefly, rhAHF was added to AHF deficient plasma and the clotting time was monitored. The activity of the rhAHF was then obtained from a calibration curve constructed using the clotting times determined from various dilutions of a lyophilized reference concentrate of known activity.

### Example 7

### Thermal Denaturation Studies of rhAHF

Because the process of physical instability is related to protein unfolding at the molecular level, our immediate objective was to analyze the rhAHF protein unfolding in detail. Thermal denaturation studies have been commonly employed to understand the structural stability of protein therapeutics and to develop a pharmaceutically stable formulation (Tsai, P. K. Volkin, D. B. et al. (1993) “Formulation design of acidic fibroblast growth factor,” Pharm Res 10(5): 649-59; Balasubramanian, S. V., Bruenn, V. J. et al. (2000) “Liposomes as formulation excipients for protein pharmaceuticals: a model protein study [In Process Citation].” Pharm Res 17(3): 344-50, which are hereby incorporated by reference in their entirety).

### Example 8

### Effect of Temperature of Secondary Structure

Temperature induced changes in rhAHF secondary structure was studied by acquiring the far-UV CD spectra (255 nm to 205 nm, FIG. 1A). At 20° C., a broad negative band at 215 nm suggested that the protein existed predominantly in β-sheet conformation. This is in agreement with the structure, proposed based on homology modeling (Pan, Y. T., DeFay, T. et al. (1995) “Proposed structure of the A domains of factor VIII by homology modelling [letter].” Nat Struct Biol 2(0): 740-4, which is hereby incorporated by reference in its entirety). As the temperature is increased over the temperature range of from 20° C. to 50° C., there were no significant changes in the far-UV CD spectrum; indicating that the secondary structure of the protein was not altered. In the temperature range of from 50° C. to 65° C., the ellipticity at 215 nm increased progressively with increasing temperature suggesting an increase in the β-sheet conformation. At temperatures over 65° C., significant changes were observed in the spectral characteristics. The CD spectra had red-shifted by approximately 2 nm and appearance of a positive band in the range from 205 nm to 210 nm range suggested the formation of anti-parallel β-strands possibly leading to formation of aggregates eventually stabilized by intermolecular β-strands (Hilbich, C., Kisters-Woike, B. et al. (1991) “Aggregation and secondary structure of synthetic amyloid beta A4 peptides of Alzheimer's disease.” J Mol Biol 218(1): 149-63; Hammarstrom, P. M., Persson, M. et al. (1999) “Structural mapping of an aggregation nucleation site in a molten globule intermediate.” J Biol Chem 274(46): 32897-903, which are hereby incorporated by reference in their entirety). Thus the secondary structure appeared to undergo the following conformational transition:

parallel-β sheet . . . increased β-sheet content . . . anti-parallel β-sheets.

Far-UV spectral data was used to calculate the Fₐₚₚ, the apparent fraction in the unfolded form, according to the method described herein.

Analysis of the profile indicated that the melting of the protein appeared to occur in two distinct stages. The first transition was sharp and prominent, occurring approximately at the range of from 60° C. to 62° C. and may possibly be associated with the melting of heavy chain of the protein while the second transition observed at about 70° C., was relatively broad. Since rhAHF is a multi-domain protein the observed multi-stage transition may be attributed to the unfolding of heavy and light chains of the rhAHF at different temperatures.

### Example 9

### Effect of Temperature on Tertiary and Secondary Structure and Evidence for the Existence of “Structured Intermediates”

While the near-UV CD spectrum is indicative of the tertiary structure, the far-UV CD spectrum is indicative of the secondary structure. We investigated the temperature dependence of the near-UV CD spectrum over the wavelength range of from 255 nm to 320 nm, (FIG. 1B). At 20° C., there were two positive peaks at about 295 nm and about 268 nm and as the temperature was increased, the intensity of the peaks decreased.

The near-UV CD spectrum was used to calculate the temperature dependence of the unfolding of tertiary structure. With increase in temperature, ellipticity at 295 nm decreased and thus Fₐₚₚ increased with midpoint of main transition occurring approximately in the range of from 50° C. to 52° C., (FIG. 1C). The temperature dependence of far-UV CD spectrum was monitored over the wavelength range of from 205 nm to 255 nm and the main transition detected by far-UV CD was considerably higher, approximately from 60° C. to 62° C., (FIG. 1C). Such a difference in the temperature at which tertiary and secondary structural changes occurred confirmed the existence of intermediate unfolded state(s). (Ptitsyn, O. B., Pain, R. H. et al. (1990) “Evidence for a molten globule state as a general intermediate in protein folding.” FEBS Lett 262(1): 20-4, which is hereby incorporated by reference in its entirety). While the multistage transition was apparent for the far-UV CD spectra, it was relatively less apparent for the near-UV CD spectra.

Factor VIII is a multi-domain protein with several tryptophan residues and changes in Trp fluorescence may provide information on gross tertiary structural changes in the protein, spanning different domains. Fluorescence emission spectra of rhAHF were acquired over the temperature range of from 25° C. to 90° C. to detect changes in Trp fluorescence, (FIG. 2). The data indicated multistage transitions and they did not overlap with the one observed by far-UV CD. The midpoint of the main transition was approximately in a range of from 50° C. to 52° C. and was almost similar to the one observed for the near-UV CD spectrum. Over the temperature range of from 20° C. to 50° C., there was an approximately 33% decrease in the intensity of the emission at 330 nm. Over the same temperature range of from 20° C. to 50° C., the far-UV CD data indicated that the secondary structure of the protein was intact suggesting the existence of a structured intermediate (SI₁) in the unfolding pathway. Between from 50° C. to 65° C., it was observed that the decrease in fluorescence intensity was approximately 44% and this temperature range was also associated with the main transition occurring at about 52° C. (near-UV CD data) indicating significant loss of tertiary structure. The far-UV CD data also showed a transition over this temperature range and indicated a corresponding increase in the β-sheet conformation suggesting the existence of the second structured intermediate (SI₂). Between from 65° C. to 90° C., a further 23% decrease in intensity was observed and the fluorescence profile indicated complete loss of tertiary structure. The far-UV CD studies above 65° C. indicated that the protein had a tendency to form intermolecular β-strands suggesting the existence of the third structure intermediate (SI₃). Thus, our initial analysis showed that there were three distinct population of intermediate structures in the unfolding pathway; SI₁ with unaltered secondary structure and small changes in the tertiary structure; SI₂ with significant loss of tertiary structure and increased β-sheet conformation and SI₃ with complete loss of tertiary structure and anti-parallel β-strands.

### Example 10

### Effects of Thermal Denaturation of the Exposure of Hydrophobic Domains

To determine whether rhAHF forms intermediate structures with exposed hydrophobic domains during the unfolding process, we monitored the complexation of the protein with ANS, a probe of hydrophobic domains (Aloj, S. M., Ingham, K. C. et al. (1973) “Interactions of 1,8 ANS with human luteinizing hormones: A probe for subunit interactions of hcg and hlh.” Arch. Biochem. Biophysics 155: 478-479; Purohit, S., Shao, K. et al. (1997) “Mutants of human choriogonadotropin lacking N-glycosyl chains in the alpha-subunit. 1. Mechanism for differential action of the N-linked carbohydrates.” Biochemistry 36(40): 12355-63; Balasubramanian, V., Nguyen, L. T. et al. (1998) “Interferon-gamma-inhibitory oligodeoxynucleotides alter the conformation of interferon-gamma.” Mol Pharmacol 53(5): 926-32, which are hereby incorporated by reference in their entirety). The fluorescence intensity of protein bound ANS increased with increasing temperature, (FIG. 3). Over the temperature range of from 25° C. to 45° C. the increase in ANS intensity was almost linear suggesting a progressive increase in the exposure of hydrophobic domains of SI₁. Over the temperature range of from 50° C. to 60° C., there was a substantial increase in intensity (about 40% increase in intensity at 65° C.) suggesting more exposure of hydrophobic domains and may be associated with SI₂. The sharp increase in fluorescence of the protein-ANS complex in the temperature range of from 85° C. to 90° C. was an indication of protein aggregation, which occurred as a result of protein conformational changes that increased the exposure of hydrophobic domains and was consistent with the formation of SI₃.

### Example 11

### Conformational Changes in the Protein and Its Implication

In order to get an insight into the conformational changes in the protein that promotes the reported aggregation process and decrease in activity, the protein was incubated at various temperatures and the changes in the far-UV CD spectrum was monitored, (FIG. 4). For the incubation study carried out at 90° C., formation of intermolecular β-strands was observed and the protein on annealing did not recover the native-like secondary structural features and the loss of native structural features was concomitant with loss of biological activity (data not shown). We speculate that the partially folded, structured intermediates (SI₃) with exposed hydrophobic domains promote aggregation and the aggregates are possibly stabilized by intermolecular β-strands resulting in loss of activity of the protein. This speculation is strongly reflected in the observed increase in ANS fluorescence at elevated temperatures. For the incubation studies at 37° C. and 47° C. that corresponded to formation of SI₁, no significant change in CD characteristics was observed, while at 60° C. that corresponded to formation of SI₂, an increase in the intensity of the band at 215 nm and a small blue shift was noted which is consistent with our previous observations.

### Example 12

### Conformational Analysis of Liposomal rhAHF

Though it has been documented that the AHF binds strongly to PS containing phospholipids in vivo not much is known about the nature of interaction and molecular characteristics of the complex between PS containing liposomes and AHF. In order to understand the nature of this interaction, preliminary spectral studies were carried out. Conformation of factor VIII in the presence of liposomes examined by far-UV CD is shown in (FIG. 5); the CD spectrum of protein in its native conformation is given for comparison, (FIG. 5). In the native state, the protein exists predominantly in beta sheet conformation. The CD spectrum of liposome-associated rhAHF displayed a less intense negative band at 215 nm and a small red shift indicating a change in the conformation of the native protein.

Change in the Trp fluorescence of the protein was also followed in the presence of the liposomes as a means to probe the conformational state of the protein. In the presence of liposomes, an increase in fluorescence intensity was observed relative to that of the protein sample in the absence of liposomes. In addition, the Trp emission spectrum was blue-shifted relative to that of the native protein, though the observed shift was very small, most likely as a result of interaction of the non-polar region of the protein with the liposomal membrane. This interaction might offer shielding for the fluorophore containing domains from the external environment and may prevent aggregation of the protein.

### Example 13

### Effect of Liposomes on Equilibrium Refolding of rhAHF

In order to get an insight into the effect of liposomes on the unfolding and refolding pathway of the protein and understand the role of intermediates, equilibrium refolding studies of factor VIII were carried out under controlled heating and recooling conditions at different temperatures in the presence and absence of liposomes.

FIG. 6A shows the melting and recooling of the secondary structure of the protein in the absence of liposomes. With increasing temperature, there were significant changes in the spectral characteristics. The melting as already described appeared to occur in two stages; a prominent transition with a Tm of from about 60° C. to about 62° C. and a very broad transition at about 70° C. The formation of intermolecular β-strands was prominent at temperatures ≧65° C. and there was a significant red shift in the far-UV CD spectrum accompanied by broadening of the 215 nm negative-band and occurrence of a positive band in the range of from 200 nm to 210 nm. On recooling the thermally denatured sample, there was no recovery of the native like secondary structure and broadened band at 215 nm was persistent. In the present of liposomes, as observed in FIG. 6B, the melting of the secondary stricture again appeared to occur in two stages. While the first transition was similar to the one observed for the native protein, the second transition occurred over a broader temperature range. This may possibly be the result of the liposomes forming a complex with the protein. On re-cooling, the denatured protein in the presence of liposomes appeared to recover few of its native-like secondary structural features, FIG. 6B. Further, fluorescence studies validated our CD data, where the protein on re-cooling in the presence of liposomes showed significant recovery in intensity from about 48% to about 50% while the free protein showed only from about 20% to about 30% recovery in fluorescence intensity (data not shown). The re-cooled protein in the presence of liposomes was also found to be marginally more active in comparison to the free protein (data not shown).

While the invention has been described with preferred embodiments, it is to be understood that variations and modifications may be resorted to as will be apparent to those skilled in the art. Such variations and modifications are to be considered within the purview and the scope of the claims appended hereto.

What is claimed is:

1. A method for complexing AHF in a dispersed medium, comprising:
a) providing an AHF protein,
b) altering the conformational state of said AHF protein to expose hydrophobic domains therein,
c) binding a stabilizer to said exposed hydrophobic domains, and
d) at least partially reversing said alteration to associate at least a portion of said protein with said stabilizer.

2. The method of claim 1, wherein said altering comprises contacting said protein with at least one of a chemical and physical perturbant.

3. The method of claim 2, wherein said chemical perturbant comprises organic solvent, urea, buffer having an acidic pH, or guandinium hydrochloride.

4. The method of claim 3, wherein said organic solvent comprises methanol, ethanol, glycerol, or ethylene glycol.

5. The method of claim 1, wherein said altering comprises contacting said protein with an ethanol-water mixture of from about 3% to about 80%.

6. The method of claim 2, wherein said physical perturbant comprises a thermal or pressure change.

7. The method of claim 1, wherein said stabilizer comprises liposome.

8. The method of claim 1, wherein said reversing comprises cooling.

9. The method of claim 1, wherein said reversing comprises solvent removal.

10. The method of claim 1, wherein said reversing comprises dialysis.

11. The method of claim 1, wherein said altering comprises unfolding said AHF protein.

12. The method of claim 1, wherein said reversing comprises refolding said AHF protein.

13. The method of claim 1, further comprising coating the surface of the liposome with a hydrophilic polymer.

14. The method of claim 13, wherein the hydrophilic polymer comprises polyethylene glycol.

15. The method of claim 1, wherein said alteration is performed to expose hydrophobic domains in the SI₃ conformational state of the AHF protein.

16. The method of claim 1, wherein above about 70% of said protein is associated with said stabilizer.

17. The method of claim 1, wherein said association comprises encapsulation of at least a portion of said protein by said stabilizer.

18. The method of claim 17, wherein said at least a portion of said protein comprises above about 0.5% of the protein molecule.

19. The method of claim 17, wherein said at least a portion of said protein comprises above about 25% of the protein molecule.

20. A dispersion system-associated AHF protein produced by the method of claim 1.

21. A pharmaceutically effective stabilized AHF dosage wherein greater than about 0.5% of the AHF molecule is encapsulated by a stabilizer.

22. The product of claim 21, wherein above about 25% of the AHF molecule is encapsulated by the stabilizer.

23. The product of claim 21, further comprising a dispersed system medium.

24. The product of claim 21, wherein said stabilizer comprises liposome.

25. The product of claim 21, wherein the association of a specific region of the protein with liposomes alters a therapeutic response of the non-associated protein.

26. The product of claim 25, wherein the therapeutic response is immunogenecity and antigenicity.
